(19) United States
(12) Patent Application Publication
(10) Pub. No.: US 2026/0021114 A1
(43) Pub. Date: Jan. 22, 2026

(54) COMPOSITION FOR LOWERING BLOOD LIPIDS AND CHOLESTEROL, PREPARATION METHOD THEREFOR, AND APPLICATION THEREOF

(71) Applicant: Health and Happiness (H&H) Hong Kong Limited, Quarry Bay (CN)

(72) Inventors: Hua Zhang, Quarry Bay (CN); Lingling Zhao, Quarry Bay (CN); Liangjing Wang, Quarry Bay (CN); Armando Beltrame, Quarry Bay (CN); Andrew Xiang, Quarry Bay (CN)

(73) Assignee: Health and Happiness (H&H) Hong Kong Limited, Quarry Bay (CN)

(21) Appl. No.: 18/996,524

(22) PCT Filed: Sep. 16, 2022
(86) PCT No.: PCT/CN2022/119213

§ 371 Date: Jan. 17, 2025

(30) Foreign Application Priority Data
Sep. 8, 2022 (CN) 202211096185.1

(51) Int. Cl. A61K 31/575; A61K 31/01; A61K 36/062; A61K 36/899; A61P 3/06

(52) U.S. Cl. CPC A61K 31/575; A61K 31/01; A61K 36/062; A61K 36/899; A61P 3/06

(57) ABSTRACT

The present invention relates to the technical field of medicines, and in particular to a composition for lowering blood lipids and cholesterol, a preparation method therefor, and an application thereof. The composition comprises the following components in parts by weight: 200-1000 parts of phytosterol or an analog thereof, 50-500 parts of red yeast rice extract, and 10-500 parts of lycopene. By means of compounding phytosterol or the analog thereof, the red yeast rice extract, and lycopene in the above composition, it was discovered that phytosterol or the analog thereof, the red yeast rice extract, and lycopene have a synergistic effect of lowering blood lipids and cholesterol, can be used to prepare products for lowering blood lipids and cholesterol, and have applications in the preparation of products for the prevention, alleviation, adjunctive treatment, or treatment of hyperlipidemia and/or hypercholesterolemia.

### TECHNICAL FIELD

The present disclosure belongs to the technical field of medicine, and in particular to a composition for lowering blood lipids and cholesterol and preparation method therefor and use thereof.

### BACKGROUND

In recent years, blood lipid levels in the population have gradually increased, and the incidence of dyslipidemia has significantly increased. The overall prevalence of dyslipidemia among adults reached as high as 40.40%, and the prevalence of hypercholesterolemia among children and adolescents has also significantly increased, with a total prevalence of 23.5% for dyslipidemia. This indicates that the prevalence of adult dyslipidemia and related disease burdens will continue to increase in the future. Dyslipidemia is an important risk factor for cardiovascular disease. Currently, cardiovascular disease has gradually become a class of diseases with the highest mortality rate worldwide, especially in developing countries. Hyperlipidemia, as one of the “THREE HIGHS”, usually refers to the increase in serum cholesterol and triglycerides, and has been found to be significantly related to cardiovascular diseases such as hypertension and atherosclerosis. Therefore, lowering blood lipids and cholesterol has become an urgent problem for patients with the “THREE HIGHS”.

### SUMMARY OF THE INVENTION

Therefore, the technical problem to be solved by the present application is to provide a composition for lowering blood lipids and cholesterol and a preparation method therefor and an use thereof.

To this end, the present application provides the following technical solutions:

A composition for lowering blood lipids and cholesterol, wherein the composition comprises the following components in parts by weight: 200 to 1000 parts by weight of phytosterol or an analog thereof, 50 to 500 parts by weight of red yeast rice extract and 10 to 500 parts by weight of lycopene.

Optionally, the composition comprises the following components in parts by weight: 400 to 800 parts by weight of phytosterol or an analog thereof, 150 to 300 parts by weight of red yeast rice extract and 50 to 200 parts by weight of lycopene.

Optionally, the composition comprises the following components in parts by weight: 754 parts by weight of phytosterol or an analog thereof, 114.9 parts by weight of red yeast rice extract and 99.5 parts by weight of lycopene: or 682 parts by weight of phytosterol or an analog thereof, 280 parts by weight of red yeast rice extract and 99 parts by weight of lycopene.

A method for preparing the composition for lowering blood lipids and cholesterol, wherein the raw materials are weighed according to the formula and mixed.

A preparation, wherein the composition for lowering blood lipids and cholesterol is used as an active ingredient.

Optionally, the preparation further comprises a pharmaceutically allowable drug excipient or carrier.

Optionally, the forms of the preparation comprise a liquid preparation and a solid preparation.

Optionally, the forms of the preparation comprise injections, tablets, capsules, powders, granules or ointments.

The composition for lowering blood lipids and cholesterol or the preparation has any one use of the following:
(1) in the manufacture of products for lowering blood lipids and/or cholesterol:
(2) in the manufacture of products for preventing, alleviating, auxiliary treating or treating hyperlipidemia and/or high cholesterol;
(3) in the manufacture of products for reducing triglycerides:
(4) in the manufacture of products for reducing hmgcra gene expression levels: and
(5) in the manufacture of products for weight loss.

Optionally, the product comprises a food, a food additive or a medicine.

Optionally, the food comprises a functional food, a health product or an ordinary food.

The technical solution of the present application has the following advantages:

1. A composition for lowering blood lipids and cholesterol provided by the present application, comprising the following components in parts by weight: 200 to 1000 parts by weight of phytosterol or an analog thereof, 50 to 500 parts by weight of red yeast rice extract and 10 to 500 parts by weight of lycopene. By means of combination of phytosterol or the analog thereof, the red yeast rice extract, and lycopene in the above composition, it was discovered that phytosterol or the analog thereof, the red yeast rice extract, and lycopene have a synergistic effect of lowering blood lipids and cholesterol, can be used to prepare products for lowering blood lipids and cholesterol, and have applications in the preparation of products for the prevention, alleviation, adjunctive treatment, or treatment of hyperlipidemia and/or hypercholesterolemia.

### DETAILED DESCRIPTION

The following examples are provided for a better understanding of the present application, and are not limited to the best embodiments, and do not limit the content and protection scope of the present application. Any product identical or similar to the present application obtained by combining with the features of other prior art shall fall within the protection scope of the present application.

Some specific experimental steps or conditions are not indicated in the examples can be carried out according to the operation or conditions of conventional experimental steps described in the literature in this field. If the manufacturer of the reagent or instrument used is not indicated, it is a conventional reagent product commercially available.

Phytosterols or the analogs thereof are commercially available products, and their product forms include but are not limited to microcapsule powders, ointments or oils of phytosterols or the analogs thereof, with a total sterol content of more than 50%, and can be purchased from any manufacturer. The phytosterols or the analogues thereof in the following examples are selected from phytosterol esters, which are purchased from BASF Corporation.

Lycopene is a commercially available product and their product forms include but are not limited to lycopene microcapsule powder, lycopene ointment, lycopene oil or lycopene crystalline powder. The lycopene content is more than 5% and can be purchased from any manufacturer. The lycopene oil in the following examples was purchased from Chenguang Biotechnology Group Co., Ltd.

Red yeast rice extract is a commercially available product and their product forms include but are not limited to red yeast rice powder, red yeast rice or red yeast rice extract liquid. The lovastatin content is more than 1% and can be purchased from any manufacturer. The red yeast rice extract in the following example and experimental examples was purchased from the red yeast rice powder of Hangzhou Shuangma Biotechnology Co., Ltd.

### Example

The formulas of the composition for lowering blood lipids and cholesterol of Examples 1-7 are as follows.

TABLE 1
Formulas of compositions for lowering blood lipids and cholesterol

| | Example | Example | Example | Example | Example | Example | Example |
|---|---|---|---|---|---|---|---|
| | 1 | 2 | 3 | 4 | 5 | 6 | 7 |
| Phytosterol | 1000 | 754 | 800 | 400 | 200 | 682 | 927.84 |
| esters (kg) | | | | | | | |
| Red yeast rice | 500 | 114.9 | 150 | 50 | 300 | 280 | 133.32 |
| extract (kg) | | | | | | | |
| Lycopene oil | 10 | 99.5 | 50 | 200 | 500 | 99 | 15 |
| (kg) | | | | | | | |

The method for preparing the composition for lowering blood lipids and cholesterol of Examples 1-7 comprises: weighing the raw materials according to the formula in Table 1 above and mixing them evenly.

### Experimental Example

### 1. Detection Material

### 1.1. Sample Preparation Information

Sample: The composition for lowering blood lipids and cholesterol of Examples 1-7, phytosterol ester alone, red yeast rice extract alone or lycopene oil alone were prepared into 100 mg/mL stock solution with DMSO (analytical grade) and stored at −20° C.

Positive control: Atorvastatin calcium tablets (hereinafter referred to as atorvastatin calcium), white tablets, batch number 202105219C, Lepu Medical, stored in a cool place and away from light. The atorvastatin calcium tablets were prepared into 11.6 mg/mL stock solution with DMSO, subpackaged and stored at −20° C.

### 1.2. Experimental Animal

Zebrafish were raised in fish farming water at 28° C. (water quality: 200 mg of instant sea salt per 1 L of reverse osmosis water was added, the conductivity was 450-550 μS/cm: pH was 6.5-8.5; hardness was 50-100 mg/L CaCO₃). The license number for the use of experimental animals is: SYXK (Zhe) 2022-0004. Feeding management meets the requirements of international AAALAC certification (certification number: 001458).

Zebrafish are melanin allele mutant translucent Albino strain zebrafish, which are reproduced through natural pair mating. Zebrafish aged 5 days after fertilization (5dpf) was used to determine the maximum detectable concentration (MTC) of lowering blood lipids and cholesterol and to evaluate its efficacy.

### 1.3. Instruments, Consumables and Reagents

Dissection microscope (SZX7, OLYMPUS, Japan); microinjector (IM300, Narishige, Japan): needle puller (PC-10, Narishige, Japan): CCD camera (VertAl, Shanghai Tusen Vision Technology Co., Ltd., China): Electric focusing continuous zoom fluorescence microscope (AZ100, Nikon, Japan); precision electronic balance (CP214, OHAUS, USA); 6-well plate (Nest Biotech, China): general PCR amplifier (T100, BIO-RAD, Singapore); fluorescence quantitative PCR instrument (CFX Connect, BIO-RAD, Singapore); high-speed refrigerated centrifuge (Heraeus Fresco17, ThermoFisher, Germany): UV-Vis spectrophotometer (Nanodrop2000, Thermo, Austria): microplate mini centrifuge (BE-6100, Haimen Qilin Bell Instrument Manufacturing Co., Ltd., China); PCR 96-well plate (Article No. MQ50801S×5, Monad Biotechnology Co., Ltd., China); and optical adhesive seal Membrane B (MSB1001, Bio-rad, USA).

Dimethyl sulfoxide (DMSO, batch number BCCD8942, Sigma, Switzerland); methylcellulose (batch number B2006074, Shanghai Aladdin Biochemical Technology Co., Ltd., China); cholesterlylBODIPY™ 542/563 C11 (cholesterol fluorescent probe, batch number 2291600, invitrogen, USA); Oil Red O (batch number SHBM5455, Sigma, USA): 1.2-propanediol (batch number 20210817, Sinopharm Chemical Reagent Co., Ltd., China); Pure egg yolk powder (batch number 20200809, Zhejiang Aige Biotechnology Co., Ltd. Company, China): D-(+)-glucose (batch number 20201105, Sinopharm Chemical Reagent Co., Ltd., China); absolute ethanol (batch number 20210107, Sinopharm Chemical Reagent Co., Ltd., China); FastQuant RT Kit (With gDNase) kit (Article No. KR106, TIANGEN, China); RNA-Quick Purification Kit (RNA Quick Purification Kit) (Article No. RN001, YiShan Biotech, China); PowerUp™ SYBR™ GreenMasterMix (Article No. A25742, and Thermo Fisher Scientific (China) Co., Ltd., China).

### 2. Detection Method

### 2.1. MTC Determination

5 dpf melanin allele mutant translucent Albino strain zebrafish were randomly selected and put in beakers, with 30 fish per beaker (experimental group). The atorvastatin calcium and samples (see Table 2 for final concentration) were administered by dissolving in water respectively, and the normal control group and the model control group were set at the same time, and the volume of each beaker was 25 mL. Except for the normal control group, all other experimental groups were given high-fat feed in water (the high-fat feed was glucose with a final concentration of 3 wt % and egg yolk powder with a final concentration of 1.5 mg/mL) to establish a zebrafish hyperlipidemia model. The samples were co-processed with high-fat feed for 15 hours (co-processed for 7.5 hours per day and kept in fish farming water at 28° C. for the rest of the day). During sample processing, the number of dead zebrafish in each experimental group was counted every day and removed in time. After treatment at 28° C. for 48 hours, the minimum toxic concentration (MTC) of atorvastatin calcium and samples to model zebrafish was determined.

### 2.2. Efficacy Evaluation of Lowering Triglyceride

5 dpf melanin allele mutant translucent Albino strain zebrafish were randomly selected and put in beakers, with 30 fish per beaker. The atorvastatin calcium and samples (see Table 3 for final concentration) were administered by dissolving in water respectively, and the normal control group and the model control group were set at the same time, and the volume of each beaker was 25 mL. Except for the normal control group, all other experimental groups were given high-fat feed in water (the high-fat feed was glucose with a final concentration of 3wt % and egg yolk powder with a final concentration of 1.5 mg/mL) to establish a zebrafish hyperlipidemia model. The samples were co-processed with high-fat feed for 15 hours (co-processed for 7.5 hours per day and kept in fish farming water at 28° C. for the rest of the day). After treatment at 28° C. for 48 hours, Oil Red O was given for whole-body fat staining. After the staining, 10 zebrafish from each experimental group were randomly selected and photographed under a dissection microscope. Image-J advanced image processing software was used to analyze and collect the data. The staining intensity of the zebrafish tail blood vessels was analyzed, and the efficacy of atorvastatin calcium and sample in lowering triglycerides was evaluated based on the statistical analysis results of this indicator. Statistical processing results are expressed as mean +SE. Statistical analysis was performed with SPSS26.0 software, p<0.05 indicated a statistically significant difference.

### 2.3. Efficacy Evaluation of Lowering Cholesterol

5 dpf melanin allele mutant translucent Albino strain zebrafish were randomly selected and put in beakers, with 30 fish per beaker. The atorvastatin calcium and samples (see Table 4 for final concentration) were administered by dissolving in water respectively, and the normal control group and the model control group were set at the same time, and the volume of each beaker was 25 mL. Except for the normal control group, all other experimental groups were given high-fat feed in water (the high-fat feed was glucose with a final concentration of 3wt % and egg yolk powder with a final concentration of 1.5 mg/mL) to establish a zebrafish hyperlipidemia model. The samples were co-processed with high-fat feed for 15 hours (co-processed for 7.5 hours per day and kept in fish farming water at 28° C. for the rest of the day). After treatment at 28° C. for 32 hours, cholesterol fluorescent probe (final concentration 1 mg/mL) was administered. After continuing the treatment for 16 hours, 10 zebrafish from each experimental group were randomly selected and photographed under a fluorescence microscope. NIS-Elements D 3.20 advanced image processing software was used to analyze and collect the data. The cholesterol fluorescence intensity of the zebrafish tail blood vessels was analyzed, and the efficacy lowering cholesterol of the samples was evaluated by the statistical analysis results of this index. Statistical processing results are expressed as mean±SE. Statistical analysis was performed with SPSS26.0 software, p<0.05 indicated a statistically significant difference.

### 2.4. Effect on the hmgcra Gene Expression Levels

5 dpf melanin allele mutant translucent Albino strain zebrafish were randomly selected and put in beakers, with 30 fish per beaker. The atorvastatin calcium and samples (see Table 7 for final concentration) were administered by dissolving in water respectively, and the normal control group and the model control group were set at the same time, and the volume of each beaker was 25 mL. Except for the normal control group, all other experimental groups were given high-fat feed in water (the high-fat feed was glucose with a final concentration of 3 wt % and egg yolk powder with a final concentration of 1.5 mg/mL) to establish a zebrafish hyperlipidemia model. The samples were co-processed with high-fat feed for 15 hours (co-processed for 7.5 hours per day). After treatment at 28° C. for 48 hours, total RNA of 2.00 μg zebrafish sample was taken, and 20.0 μL cDNA was synthesized according to the instructions of the cDNA first chain synthesis kit. The expression of β-actin and hmgcra genes was detected by q-PCR (see Table 6 for the sequence of primers). The RNA phase of hmgcra gene was calculated using 8-actin as the internal reference of gene expression. Statistical processing results are expressed as mean +SE. Statistical analysis was performed with SPSS26.0 software, p<0.05 indicated a statistically significant difference.

### 3. Determination Results

### 3.1. MTC

Under the conditions of this experiment, the MTC of atorvastatin calcium on model zebrafish is 11.6 μg/mL. See table below for details.

TABLE 2
MTC experiment results of samples (n = 30)

| | | Number of | Death | |
|---|---|---|---|---|
| | Concentration | deaths | rates | |
| Group | (μg/mL) | (fish) | (%) | Phenotype |
| Normal control | — | 0 | 0 | No obvious |
| group | | | | abnormality |
| Model control | — | 0 | 0 | No obvious |
| group | | | | abnormality |
| Atorvastatin | 11.6 | 0 | 0 | Similar to the model |
| calcium | | | | control group |
| Example 1 | — | 0 | 0 | Similar to the model |
| | | | | control group |
| Example 2 | — | 0 | 0 | Similar to the model |
| | | | | control group |
| Example 3 | — | 0 | 0 | Similar to the model |
| | | | | control group |
| Example 4 | — | 0 | 0 | Similar to the model |
| | | | | control group |
| Example 5 | — | 0 | 0 | Similar to the model |
| | | | | control group |
| Example 6 | | 0 | 0 | Similar to the model |
| | | | | control group |
| Example 7 | — | 0 | 0 | Similar to the model |
| | | | | control group |
| Phytosterol | — | 0 | 0 | Similar to the model |
| esters | | | | control group |
| Red yeast rice | — | 0 | 0 | Similar to the model |
| extract | | | | control group |
| Lycopene oil | — | 0 | 0 | Similar to the model |
| | | | | control group |

### 3.2. Efficacy Evaluation of Lowering Triglyceride

The results are shown in the table below.

TABLE 3
Evaluation of efficacy of lowering triglyceride

| | Concentration | Triglyceride content (Unit |
|---|---|---|
| Group | (μg/mL) | in pixels, mean ± SE) |
| Normal control group | — | 4877 ± 161***^{a} |
| Model control group | — | 17812 ± 487 |
| Atorvastatin calcium | 11.6 | 11104 ± 588***^{c} |
| Example 1 | 500 | 7500 ± 298***^{b} |
| Example 2 | 500 | 6393 ± 284***^{b} |
| Example 3 | 500 | 9525 ± 463***^{c} |
| Example 4 | 500 | 8276 ± 574***^{c} |
| Example 5 | 500 | 7706 ± 355***^{c} |
| Example 6 | 500 | 8956 ± 353***^{c} |
| Example 7 | 500 | 7495 ± 475***^{c} |
| Phytosterol esters | 1000 | 13224 ± 632** |
| Red yeast rice extract | 1000 | 14534 ± 307* |
| Lycopene oil | 1000 | 13035 ± 316** |

Note:
Compared with the model control group, *P < 0.05, **P < 0.01, ***P < 0.001, and different letters indicated significant differences (P < 0.05).

As can be seen from the above table, the positive control group, the composition for lowering blood lipids and cholesterol of Examples 1-7 of the present application, and phytosterol esters alone, red yeast rice extract alone or lycopene oil alone all were able to significantly lower triglycerides compared with the model control group, P<0.05. Compared with the phytosterol esters alone, the red yeast rice extract alone or the lycopene oil alone, the composition for lowering blood lipids and cholesterol of Examples 1-7 have significant differences in lowering triglycerides, indicating that composition for lowering blood lipids and cholesterol of the present application can synergistically reduce triglycerides.

### 3.3. Efficacy Evaluation of Lowering Cholesterol

The results are shown in the table below.

TABLE 4
Evaluation of efficacy of lowering cholesterol

| | Concentration | Cholesterol content (Unit |
|---|---|---|
| Group | (μg/mL) | in pixels, mean ± SE) |
| Normal control group | — | 75024 ± 7200*** |
| Model control group | — | 175509 ± 10840 |
| Atorvastatin calcium | 11.6 | 122765 ± 11371* |
| Example 1 | 500 | 120490 ± 10802* |
| Example 2 | 500 | 127722 ± 9318** |
| Example 3 | 500 | 121410 ± 9071** |
| Example 4 | 500 | 128770 ± 9971* |
| Example 5 | 500 | 129648 ± 8947* |
| Example 6 | 500 | 128554 ± 10647* |
| Example 7 | 500 | 124534 ± 9473* |
| Phytosterol esters | 1000 | 144720 ± 9155 |
| Red yeast rice extract | 1000 | 153726 ± 9780 |
| Lycopene oil | 1000 | 141239 ± 10264 |

Note:
Compared with the model control group, *P < 0.05, **P < 0.01, ***P < 0.001.

As can be seen from the above table,, the positive control group, the composition for lowering blood lipids and cholesterol of Examples 1-7 of the present application were able to significantly lower cholesterol compared with the model control group, P<0.05. Compared with the phytosterol esters alone, the red yeast rice extract alone or the lycopene oil alone, the composition for lowering blood lipids and cholesterol of Examples 1-7 have significant differences in lowering triglycerides, indicating that composition for lowering blood lipids and cholesterol of the present application can synergistically reduce cholesterol.

### 3.4. Effect on the hmgcra Gene Expression Levels

At the end of the experiment, total RNA of zebrafish was extracted, and the concentration of RNA and the A₂₆₀/A₂₈₀ ratio were measured using a UV-visible spectrophotometer (Table 5). The A₂₆₀/A₂₈₀ ratios were both between 1.8 to 2.2. indicating that the extracted total RNA of zebrafish is of good quality and can be used for subsequent q-PCR experiments. The primer sequences arc shown in Table 6.

TABLE 5
Total RNA concentration and A₂₆₀/A₂₈₀ ratio (n = 30)

| | | RNA concentration (μg/μL) | | | A₂₆₀/A₂₈₀ | | |
|---|---|---|---|---|---|---|---|
| | Concentration | Sample | Sample | Sample | Sample | Sample | Sample |
| Group | (μg/mL) | 1 | 2 | 3 | 1 | 2 | 3 |
| Normal | — | 0.786 | 0.596 | 0.553 | 1.95 | 1.97 | 1.96 |
| control | | | | | | | |
| group | | | | | | | |
| Model | — | 0.397 | 0.480 | 0.452 | 1.95 | 1.94 | 1.94 |
| control | | | | | | | |
| group | | | | | | | |
| Atorvastatin | 11.6 | 0.346 | 0.499 | 0.487 | 1.98 | 1.96 | 1.95 |
| calcium | | | | | | | |
| Example 1 | 500 | 0.721 | 0.721 | 0.721 | 1.96 | 1.93 | 1.96 |
| Example 2 | 500 | 0.678 | 0.670 | 0.527 | 2.01 | 2.02 | 1.96 |
| Example 3 | 500 | 0.721 | 0.365 | 0.384 | 1.88 | 1.99 | 1.95 |
| Example 4 | 500 | 0.400 | 0.785 | 0.447 | 1.89 | 1.97 | 1.90 |
| Example 5 | 500 | 0.784 | 0.529 | 0.575 | 1.96 | 1.89 | 1.99 |
| Example 6 | 500 | 0.654 | 0.643 | 0.531 | 1.90 | 1.96 | 1.95 |
| Example 7 | 500 | 0.595 | 0.630 | 0.716 | 1.98 | 1.91 | 1.94 |
| Phytosterol | 1000 | 0.420 | 0.714 | 0.574 | 1.90 | 1.95 | 1.91 |
| esters | | | | | | | |
| Red yeast | 1000 | 0.549 | 0.686 | 0.734 | 1.96 | 1.95 | 2.00 |
| rice extract | | | | | | | |
| Lycopene | 1000 | 0.427 | 0.458 | 0.408 | 1.94 | 1.90 | 1.92 |
| oil | | | | | | | |

TABLE 6
Primer sequence information

| Gene | Primer sequence | |
|---|---|---|
| β-actin | Forward primer | 5′-TCGAGCAGGAGATGGG |
| | (SEQ ID NO. 1) | AACC-3′ |
| | Reverse primer | 5′-CTCGTGGATACCGCAA |
| | (SEQ ID NO. 2) | GATTC-3′ |
| hmgcra | Forward primer | 5′-CACATGACTCACAACA |
| | (SEQ ID NO. 3) | GATCAAAAG-3′ |
| | Reverse primer | 5′-GAGAAGAAGGGATCGG |
| | (SEQ ID NO. 4) | TTGC-3′ |

The results are shown in the table below.

TABLE 7
Relative expression of hmgcra gene

| | Concentration | Relative expression of |
|---|---|---|
| Group | (μg/mL) | hmgcra gene (mean ± SE) |
| Normal control group | — | 0.764 ± 0.015***^{c} |
| Model control group | — | 1 ± 0.12 |
| Atorvastatin calcium | 11.6 | 0.542 ± 0.003***^{a} |
| Example 1 | 500 | 0.616 ± 0.006***^{a} |
| Example 2 | 500 | 0.464 ± 0.02***^{a} |
| Example 3 | 500 | 0.568 ± 0.01***^{a} |
| Example 4 | 500 | 0.648 ± 0.013***^{b} |
| Example 5 | 500 | 0.637 ± 0.011***^{b} |
| Example 6 | 500 | 0.575 ± 0.012***^{a} |
| Example 7 | 500 | 0.593 ± 0.012***^{a} |
| Phytosterol esters | 1000 | 0.803 ± 0.053* |
| Red yeast rice extract | 1000 | 0.784 ± 0.062** |
| Lycopene oil | 1000 | 0.898 ± 0.054* |

Note:
Compared with the model control group, *P < 0.05, **P < 0.01, ***P < 0.001, and different letters indicated significant differences (P < 0.05).

As can be seen from the above table, the positive control group, the composition for lowering blood lipids and cholesterol of Examples 1-7 of the present application, and phytosterol esters alone, red yeast rice extract alone or lycopene oil alone all can significantly reduce the hmgcra gene expression levels compared with the model control group, P<0.05. Compared with the phytosterol esters alone, the red yeast rice extract alone or the lycopene oil alone, the composition for lowering blood lipids and cholesterol of Examples 1-7 have significant differences in reducing the relative expression of the hmgcra gene, indicating that composition for lowering blood lipids and cholesterol of the present application can synergistically reduce relative expression of the hmgcra gene.

Obviously, the above-mentioned embodiments are only examples for clear description, and are not intended to limit the implementation manner. For those of ordinary skill in the art, changes or modifications in other different forms can also be made on the basis of the above description. There is no need and cannot be exhaustive of all implementations here. And the obvious changes or changes derived from this are still within the protection scope of the present application.

1. A composition for lowering blood lipids and cholesterol, wherein the composition comprises the following components in parts by weight: 200 to 1000 parts by weight of phytosterol or an analog thereof, 50 to 500 parts of red yeast rice extract and 10 to 500 parts by weight of lycopene.

2. The composition for lowering blood lipids and cholesterol according to claim 1, wherein the composition comprises the following components in parts by weight: 400 to 800 parts by weight of phytosterol or an analog thereof, 150 to 300 parts by weight of red yeast rice extract and 50 to 200 parts by weight of lycopene.

3. The composition for lowering blood lipids and cholesterol according to claim 1, wherein the composition comprises the following components in parts by weight: 754 parts by weight of phytosterol or an analog thereof, 114.9 parts by weight of red yeast rice extract and 99.5 parts by weight of lycopene; or 682 parts by weight of phytosterol or an analog thereof, 280 parts by weight of red yeast rice extract and 99 parts by weight of lycopene.

4. A method for preparing the composition for lowering blood lipids and cholesterol according to claim 1, wherein the raw materials are weighed according to the formula and mixed.

5. A preparation, wherein the composition for lowering blood lipids and cholesterol according to claim 1 is used as an active ingredient.

6. The preparation according to claim 5, further comprising a pharmaceutically allowable drug excipient or carrier.

7. A method for lowering blood lipids and/or cholesterol, comprising administering the composition for lowering blood lipids and cholesterol according to claim 1 to a subject in need.

8. (canceled)

9. (canceled)

10. A method for preventing, alleviating, auxiliary treating or treating hyperlipidemia and/or high cholesterol, comprising administering the composition for lowering blood lipids and cholesterol according to claim 1 to a subject in need.

11. A method for reducing triglycerides, comprising administering the composition for lowering blood lipids and cholesterol according to claim 1 to a subject in need.

12. A method for reducing hmgcra gene expression levels, comprising administering the composition for lowering blood lipids and cholesterol according to claim 1 to a subject in need.

13. A method for weight loss, comprising administering the composition for lowering blood lipids and cholesterol according to claim 1 to a subject in need.

14. A method for lowering blood lipids and/or cholesterol, comprising administering the preparation according to claim 5 to a subject in need.

15. A method for preventing, alleviating, auxiliary treating or treating hyperlipidemia and/or high cholesterol, comprising administering the preparation according to claim 5 to a subject in need.

16. A method for reducing triglycerides, comprising administering the preparation according to claim 5 to a subject in need.

17. A method for reducing hmgcra gene expression levels, comprising administering the preparation according to claim 5 to a subject in need.

18. A method for weight loss, comprising administering the preparation according to claim 5 to a subject in need.

19. The preparation according to claim 5, wherein the forms of the preparation comprise a liquid preparation and a solid preparation.

20. The preparation according to claim 5, wherein the forms of the preparation comprise injections, tablets, capsules, powders, granules or ointments.

21. A method for preparing the composition for lowering blood lipids and cholesterol according to claim 2, wherein the raw materials are weighed according to the formula and mixed.

22. A preparation, wherein the composition for lowering blood lipids and cholesterol according to claim 2 is used as an active ingredient.


SEQUENCE LISTING (4 sequences)

SEQ ID NO: 1 - DNA, 20 bp, synthetic construct
  source 1..20: mol_type = other DNA
tcgagcaggagatgggaacc

SEQ ID NO: 2 - DNA, 21 bp, synthetic construct
  source 1..21: mol_type = other DNA
ctcgtggataccgcaagattc

SEQ ID NO: 3 - DNA, 25 bp, synthetic construct
  source 1..25: mol_type = other DNA
cacatgactcacaacagatcaaaag

SEQ ID NO: 4 - DNA, 20 bp, synthetic construct
  source 1..20: mol_type = other DNA
gagaagaagggatcggttgc